(19) United States
(12) Patent Application Publication
(10) Pub. No.: US 2012/0197234 A1
(43) Pub. Date: Aug. 2, 2012

(54) DELIVERY METHODS FOR A BIOLOGICAL PACEMAKER MINIMIZING SOURCE-SINK MISMATCH

(75) Inventor: Vinod Sharma, Maple Grove, MN (US)

(73) Assignee: Medtronic, Inc., Minneapolis, MN (US)

(21) Appl. No.: 13/109,151

(22) Filed: May 17, 2011

Related U.S. Application Data
(60) Provisional application No. 61/437,941, filed on Jan. 31, 2011.

(51) Int. Cl. A61M 25/06; A61M 5/32; A61M 31/00

(52) U.S. Cl. 604/511; 604/522; 604/272

(57) ABSTRACT

The present invention includes methods, systems, devices, and apparatus relating to minimizing source-sink mismatch associated with the application of a biological pacemaker in cardiac tissue. Stable and robust pacemaker activity is achieved by the application of the biological pacemaker at more than one site, in a linear pattern.

### CONTINUING APPLICATION DATA

This application claims the benefit of U.S. Provisional Application Ser. No. 61/437,941, filed Jan. 31, 2011, which is incorporated by reference herein.

### BACKGROUND

Cardiac contraction in a healthy human heart is initiated by spontaneous excitation of the sinoatrial (“SA”) node, which is located in the right atrium. The electric impulse generated by the SA node travels to the atrioventricular (“AV”) node where it is transmitted to the brindle of His and to the Purkinje network. The fibers in the Purkinje network branch out in many directions to facilitate coordinated contraction of the left and right ventricles, thus providing natural pacing. In some disease states, the heart loses some of its natural capacity to pace properly. Such dysfunction is commonly treated by implanting a pacemaking device that generates an electronic pulse.

While effectively improving the lives of many patients, such implantable pacemakers have certain technical limitations. For example, implantable pacemakers rely on a self-contained power source such as a battery and consequently have a limited lifetime before the power source is in need of replacement. This is particularly problematic in individuals who develop pacing dysfunction at a younger age. Hence, an otherwise healthy patient may require multiple surgeries to replace the power source or the entire implantable pacemaker. In addition, implantable pacemaker batteries are large and are usually the bulkiest pacemaker component. A pacemaker's size and capability for implantation in different body regions are typically dictated by the battery size. Also, implantable pacemakers have very limited or no capacity for directly responding to the body's endogenous signaling the way the SA node responds to such signaling, i.e. by a modulation of the heart rate relative to the physiological and emotional state (e.g. sleep, rest, stress, exercise). Electronic pacemakers are also unable to mimic normal respiratory and autonomic tone mediated beat-to-beat variability in heart rate (heart rate variability), which are hypothesized to have impact on overall body homeostasis.

Biological pacemakers implemented using gene or cell based therapies present great potential and promise as therapeutic alternatives to implantable electronic pacemakers for the treatment of cardiac disorders. Several efforts have been undertaken to create an artificial site in the heart that can mimic the pacemaking function of the SA node. See, for example, Qu et al., 2003, Circulation; 107(8):1106-9; Bucchi et al., 2006, Circulation; 114(10):992-9; Tse et al., 2006, Circulation; 114(10):1000-11; Kashiwakura et al., Circulation; 114(16):1682-6, and Jaye et al., 2010, Circulation; 122(21-supplement):A21428.

However, studies have yet to demonstrate the establishment of a biological pacemaker with long term stability. This variation in data and biopacemaking activity may be because these investigators overlooked something critical in the design of the biological pacemaker—the source-sink mismatch. With source-sink mismatch, if the tissue load on the regions driving the excitation (i.e. pacemaker region) is large relative to pacemaker's size, the pacemaker is unable to drive the tissue in a stable and reproducible manner. And, to achieve reliable excitation of the load tissue and produce stable pacemaking, the pacemaker regions must be sufficiently large and of correct geometry relative to the tissue architecture (e.g. fiber direction).

There is a need for improved methods, systems, and apparatus for the delivery of gene or cell based therapies for the establishment of biological pacemakers in the treatment of cardiac disorders that minimize source-sink mismatch, resulting in a stable robust biological pacemaker that more closely recreates the normal physiological pacemaking function of the SA node.

### SUMMARY OF THE INVENTION

The present invention includes a method for delivering a biological pacemaker agent affecting cardiac pacing of the heart, the method including delivering the biological pacemaker agent affecting cardiac pacing at two or more sites in the heart; delivery sites located so that the injectate from one delivery site overlaps with the injectate from neighboring injection sites; and the delivery sites forming a linear pattern. In some aspects, delivery is perpendicular to the fibers of the heart. In some aspects, delivery is parallel to the fibers of the heart. In some aspects, delivery is at an angle to the fibers of the heart.

The present invention includes a method of establishing an artificial pacemaker in cardiac tissue, the method including delivering a biological pacemaker agent affecting cardiac pacing at two or more sites in the heart; each delivery site located so that the injectate from one delivery site overlaps with the injectate from neighboring injection sites; and the delivery sites forming a linear pattern. In some aspects, delivery is perpendicular to the fibers of the heart. In some aspects, delivery is parallel to the fibers of the heart. In some aspects, delivery is at an angle to the fibers of the heart.

The present invention includes a method of providing an exogenous biopacemaker to cardiac tissue, the method including delivering a biological pacemaker agent affecting cardiac pacing at two or more sites in the heart; each delivery site located so that the injectate from one delivery site overlaps with the injectate from neighboring injection sites; and the delivery sites forming a linear pattern. In some aspects, delivery is perpendicular to the fibers of the heart. In some aspects, delivery is parallel to the fibers of the heart. In some aspects, delivery is at an angle to the fibers of the heart.

The present invention includes a method for treating a cardiac pacing condition in a subject, the method including administering a biological pacemaker agent affecting cardiac pacing at two or more sites in the subject's heart; each delivery site located so that the injectate from one delivery site overlaps with the injectate from neighboring injection sites; and the delivery sites forming a linear pattern. In some aspects, delivery is perpendicular to the fibers of the heart. In some aspects, delivery is parallel to the fibers of the heart. In some aspects, delivery is at an angle to the fibers of the heart.

The present invention includes a method of delivering an intervention affecting cardiac pacing to the heart, the method including delivering the intervention affecting cardiac pacing at two or more sites in the heart; each delivery site located so that the injectate from one delivery site overlaps with the injectate from neighboring injection sites; and the delivery sites forming a linear pattern. In some aspects, delivery is perpendicular to the fibers of the heart. In some aspects, delivery is parallel to the fibers of the heart. In some aspects, delivery is at an angle to the fibers of the heart.

In some aspects of the methods and systems of the present invention, the delivery of the intervention or biological pacemaker agent effecting cardiac pacing is into cardiac atrial cells or cardiac ventricle cells.

In some aspects of the methods and systems of the present invention, each delivery site is located less than about 10 millimeters (mm) from any other delivery site. In some aspects of the methods and systems of the present invention, each delivery site is located about 2 mm to about 5 mm from any other delivery site. In some aspects of the methods and systems of the present invention, each delivery site is located about 3 mm to about 6 mm from any other delivery site.

In some aspects of the methods and systems of the present invention, the delivery or administration of the intervention or biological pacemaker agent affecting cardiac pacing includes two, three, four, five, six, or more delivery sites within the heart.

In some aspects of the methods and systems of the present invention, the intervention or biological pacemaker agent effecting cardiac pacing includes gene therapy, cell therapy, ablation, and/or drug delivery. In some aspects, the intervention or biological pacemaker agent effecting cardiac pacing includes cell therapy. In some aspects, cell therapy includes stem cell therapy or genetically modified cell therapy.

In some aspects of the methods and systems of the present invention, the intervention or biological pacemaker agent effecting cardiac pacing includes an exogenous polynucleotide encoding a membrane polypeptide that regulates the flow of ions across a cell membrane. In some aspects, the polynucleotide is present in a vector. In some aspects, the vector includes a viral vector, a transposon vector, and/or a plasmid vector. In some aspects, the viral vector includes a single strand adeno-associated virus or a self complementary adeno-associated virus.

In some aspects of the methods and systems of the present invention, the exogenous polynucleotide encoding a membrane polypeptide that regulates the flow of ions across a cell membrane is present in a genetically modified cell.

In some aspects of the methods and systems of the present invention, the membrane polypeptide that regulates the flow of ions across a cell membrane is an ion channel.

In some aspects of the methods and systems of the present invention, the ion channel includes a calcium channel, a sodium channel, a chloride channel, SERCA2a, a non-specific leak channel, or a potassium channel.

In some aspects of the methods and systems of the present invention, the ion channel includes a potassium channel. In some aspects, the potassium channel includes a member of the Kv1, Kv2, Kv3, Kv4, Kv5, Kv6, Kv7, Kv8, and/or Kv9 family. In some aspects, the potassium channel includes Kv1.3.

In some aspects of the methods and systems of the present invention, the ion channel includes a hyperpolarization-activated cyclic nucleotide-gated (HCN) channel. In some aspects, the hyperpolarizaton-activated cyclic nucleotide-gated (HCN) channel includes HCN1, HCN2, HCN3, and/or HCN4. In some aspects, the hyperpolarizaton-activated cyclic nucleotide-gated (HCN) channel includes HCN4. In some aspects, the amino acid sequence of the encoded HCN polypeptide includes one, two, three, four, or more mutations. In some aspects, the amino acid sequence of the HCN polypeptide includes a truncation.

In some aspects, delivering is with the use of a needle. In some aspects, delivering is by injection. In some aspects, delivering is by the use of a catheter. In some aspects, delivering includes epicardial delivery. In some aspects, delivering includes endocardial delivery.

In some aspects of the method, the biological pacemaker agent affecting cardiac pacing is delivered with a needle including two, three, or more openings, the openings having a periodic spacing along the distal end of the needle, and the biological pacemaker agent affecting cardiac pacing is delivered through all needle openings simultaneously. In some aspects, the periodic spacing is less than about 10 mm. In some aspects, the periodic spacing is about 2 mm to about 5 mm. In some aspects, the periodic spacing is about 3 mm to about 6 mm.

In some aspects of the method, the biological pacemaker agent affecting cardiac pacing is delivered with a needle including a single opening at the distal end of the needle, the biological agent pacemaker affecting cardiac pacing is repeatedly delivered through the distal end opening of the needle, the needle is withdrawn or advanced between each delivery, and delivery is in a linear pattern. The delivery sites may have a periodic spacing. In some aspects, the periodic spacing is less than about 10 mm. In some aspects, the periodic spacing is about 2 mm to about 5 mm. In some aspects, the periodic spacing is about 3 mm to about 6 mm.

In some aspects, the method further includes the use of image guidance technology to record the site(s) of delivery in cardiac tissue. In some aspects, the method further includes recording electrical impedance to determine that the needle remains located within myocardial tissue.

The present invention includes a catheter system for the delivery of a biological pacemaker agent affecting cardiac pacing to two or more sites in the heart, the catheter system including a catheter suitable for endocardial access and a needle suitable for delivery to two or more sites in the heart, the needle extendable through a distal tip of the catheter. In some aspects of the catheter system, the needle includes two or more openings, the openings having a periodic spacing. In some aspects, the periodic spacing is less than about 10 mm. In some aspects, the periodic spacing is about 2 mm to about 5 mm. in some aspects, the periodic spacing is about 3 mm to about 6 mm. In some aspects of the catheter system, the needle includes a single opening at the distal end of the needle. In some aspects, the catheter system further includes an electrode for recording electrical impedance indicating that the needle remains located within myocardial tissue. In some aspects, the catheter system further includes image guidance technology to record the site of delivery of each biological pacemaker agent affecting cardiac pacing in cardiac tissue. The present invention includes a system for the delivery of a biological pacemaker agent affecting cardiac pacing to two or more sites in the heart in a linear pattern, the system including a catheter suitable for endocardial access that includes a catheter body that defines an inner lumen and a needle for placement within the inner catheter lumen, the needle delivering a fluid including a biological pacemaker agent affecting cardiac pacing to two or more sites in the heart, each delivery site having a periodic spacing and the delivery sites forming a linear pattern. In some aspects, delivery is perpendicular to the fibers of the heart. In some aspects, delivery is parallel to the fibers of the heart. In some aspects, delivery is at an angle to the fibers of the heart. In some aspects, the periodic spacing is less than about 10 mm. In some aspects, the periodic spacing is about 2 mm to about 5 mm. In some aspects, the periodic spacing is about 3 mm to about 6 mm. In some aspects, the system further includes a fluid including a biological pacemaker agent affecting cardiac pacing. In some aspects, the system further includes at least one electrode for recording electrical impedance indicating that the needle remains located within myocardial tissue. In some aspects, the system further includes image guidance technology to record the site of delivery of each biological pacemaker effecting cardiac pacing in cardiac tissue. In some aspects, the needle includes two or more openings, the openings having a periodic spacing of 10 mm or less along the distal end of the needle. In some aspects, the needle includes single opening at the distal end of the needle. In some aspects, multiple, separate needles are use, for example, two, three, four, five, six or more needles. Such multiple needles may be enclosed in a single housing unit.

The term “and/or” means one or all of the listed elements or a combination of any two or more of the listed elements.

Unless otherwise specified, “a,” “an,” “the,” and “at least one” are used interchangeably and mean one or more than one.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list. The following detailed description of the invention is merely exemplary in nature and is not intended to limit the invention or the application and uses of the invention. Furthermore, there is no intention to be bound by any theory presented in the preceding background of the invention or the following detailed description of the invention.

### BRIEF DESCRIPTION OF THE FIGURES

FIG. 1 demonstrates dispersion of gene expression in left ventricle after a single injection of ˜100 μl biologic volume. The white bar that depicts the needle is 10 mm long.

FIGS. 2A and 2B demonstrate the spatial extent of the SA node in the rabbit. FIG. 2A is a schematic diagram of dorsal view of rabbit heart showing location and extent of central (darker gray) and peripheral (lighter gray) sinus node tissue. White dot indicates leading pacemaker site. Ao indicates aorta; CS, coronary sinus; PA, pulmonary artery; PV, pulmonary vein; RA, right atrium; RV, right ventricle; IVC, inferior vena cava; and SVC, superior vena cava. See also Dobrzynski et al., 2005, Circulation; 111:846-854. FIG. 2B is a photograph of a rabbit right atrial preparation on which leading pacemaker sites in the RA region are schematically shown by black dots. Black dotted lines represent borders of block zone in septum region. CS, coronary sinus. See also Fedorov et al., 2006, Am J Physiol Heart Circ Physiol; 291(2):H612-23.

FIGS. 3A and 3B present the pattern of injections. FIG. 3A presents pattern of injection in the left ventricle (LV). FIG. 3B presents the pattern of injection in the left atrium (LA). LAA refers to left atrial appendage. The linear injection pattern is perpendicular to the fiber direction of the heart.

FIGS. 4A to 4D present the results from three AV node ablated canines in which the left ventricle was injected with a HCN4 pacemaker gene construct using a linear injection. Biologic dose is in pfu of viral particles. In FIG. 4A the biologic dose was 1.3×10¹⁰ pfu of HCN4. In FIG. 4B the biologic dose was 1.3×10¹⁰ pfu of HCN4tr. In FIG. 4C the biologic dose was 6.6×10⁸ pfu of HCN4tr. FIG. 4D shows the expression of the HCN protein detected using immunohistochemical techniques.

FIGS. 5A and 5B show a catheter system with three needles, spaced for linear injections. In FIG. 5A the needles are retracted. In FIG. 5B the needles have been deployed into tissue.

### DETAILED DESCRIPTION OF ILLUSTRATIVE EMBODIMENTS OF THE PRESENT INVENTION

Biological interventions for the treatment of cardiac disorders present great potential and promise. The present invention includes methods, systems, devices, and apparatus allowing for the establishment of a robust and stable artificial pacemaker site in the heart, in a manner that addresses the limitations to success presented by source-sink mismatch and in a manner that mimics the normal pacemaking function of the sinoatrial (SA) node. The SA node, also referred to herein as sinus node, SN, or sinoauricular node, originates the electrical impulse for the entire conduction system of the heart and is considered the natural pacemaker of the heart. The methods, systems, devices, and apparatus of the present invention control the number of delivery sites, the spacing between delivery sites, and/or the geometry of the location of the delivery sites with delivery of exogenous biological pacemaker agents providing for the establishment of a biological pacemaker. While in some embodiments application to cardiac tissue of the heart is preferred, the methods, systems, devices, and apparatus described herein may be applied to any of a variety of organs and tissues, including, but not limited to cardiac tissue, nervous tissue, skeletal muscle, smooth muscle, secretory epithelial tissue, beta cells of the pancreas, heart, brain, spine, nerves, lung, bladder, stomach, and blood vessels, including veins and arteries. A tissue may be heterologous tissue, including both excitable cells and nonexcitable cells. A tissue may be an excitable tissue. As used herein, excitable cells and tissues demonstrate electrical activity.

With the present invention, an exogenous biological pacemaker agent is delivered to multiple sites in the heart, or other excitable tissue. Delivery is to more than one site. For example, delivery may be to two, three, four, five, six, seven, eight, nine, ten, or more sites. For example, delivery may be to two or more, three or more, four or more, five or more, six or more, seven or more, eight or more, nine or more, or ten or more sites. For example, delivery may be to about two to about ten, about two to about nine, about two to about eight, about two to about seven, about two to about six, about two to about five, about two to about four, about two to about three, about three to about ten, about three to about nine, about three to about eight, about three to about seven, about three to about six, about three to about five, about three to about four, about four to about ten, about four to about nine, about four to about eight, about four to about seven, about four to about six, about four to about five, about five to about ten, about five to about nine, about five to about eight, about five to about seven, about five to about six, about six to about ten, about six to about nine, about six to about eight, about six to about seven, about seven to about ten, about seven to about nine, about seven to about eight, about eight to about ten, about eight to about nine, or about nine to about ten sites.

As described herein, a biological pacemaker agent may be delivered to two or more sites in a tissue, such as the myocardial tissue of the heart, so that the delivery sites form a linear pattern in the tissue. In some applications, delivery may to multiple sites so that more than one linear pattern is formed, for example, forming two, three, four, five, six, seven, eight, nine, ten, or more parallel, linear patterns in tissue. A biological pacemaker agent may be delivered to two or more sites in the heart so that the delivery sites form a linear pattern that is perpendicular to the fibers of the heart or other tissue. A biological pacemaker agent may be delivered to two or more sites in the heart so that the delivery sites form a linear pattern that is parallel to the fibers of the heart or other tissue. A biological pacemaker agent may be delivered to two or more sites in the heart so that the delivery sites form a linear pattern that is at an angle to the fibers of the heart or other tissue, for example, about a thirty degree angle, about a forty-five degree angle, about a sixty degree angle, or about a ninety degree angle.

The wall of the heart is composed of a thick layer of cardiac muscle, also called the myocardium. Cardiac muscle, like skeletal muscle, is striated, with the individual muscle cells organized in fibers. Fibers are bound together by connective tissue to form the organized architecture of the heart. The orientation of the cardiac myofibers of the heart is generally known and can be mapped using various methods, including, but not limited to, any of those described by Reese et al., 1995, Magn Reson Med; 34(6):786-91; Scollan et al., 2000, Biomed Eng; 28(8):934-44; Geerts et al., 2002, Am J Physiol Heart Circ Physiol; 283:H139-H145; or Wu et al., 2007, Magn Reson Imaging; 25(7):1048-57.

The two or more delivery sites may be closely spaced. Deliver sites are spaced so that as the injectate disperses from a given delivery site it overlaps with the biological agent dispersing from neighboring delivery sites, forming a single mass of biological pacemaker agent. For example, delivery sites may be spaced about 2 millimeters (mm) apart, about 3 mm apart, about 4 mm apart, about 5 mm apart, about 6 mm apart, about 7 mm apart, about 8 mm apart, about 9 mm apart, about 10 mm apart, about 12 mm apart, about 15 mm apart, or about 20 mm apart. Delivery sites may be spaced less than about 2 millimeters (mm) apart, less than about 3 mm apart, less than about 4 mm apart, less than about 5 mm apart, less than about 6 mm apart, less than about 7 mm apart, less than about 8 mm apart, less than about 9 mm apart, less than about 10 mm apart, less than about 12 mm apart, less than about 15 mm apart, or less than about 20 mm apart. Delivery sites may be spaced about 1 mm to about 10 mm apart, about 2 mm to about 10 mm apart, about 3 mm to about 10 mm apart, about 4 mm to about 10 mm apart, about 5 mm to about 10 mm apart, about 6 mm to about 10 mm apart, about 7 mm to about 10 mm apart, about 8 mm to about 10 mm apart, about 9 mm to about 10 mm apart, about 1 mm to about 9 mm apart, about 1 mm to about 8 mm apart, about 1 mm to about 7 mm apart, about 1 mm to about 6 mm apart, about 1 mm to about 5 mm apart, about 1 mm to about 4 mm apart, about 1 mm to about 3 mm apart, about 1 mm to about 2 mm apart, about 2 mm to about 9 mm apart, about 2 mm to about 8 mm apart, about 2 mm to about 7 mm apart, about 2 mm to about 6 mm apart, about 2 mm to about 5 mm apart, about 2 mm to about 4 mm apart, about 2 mm to about 3 mm apart, about 3 mm to about 9 mm apart, about 3 mm to about 8 mm apart, about 3 mm to about 7 mm apart, about 3 mm to about 6 mm apart, about 3 mm to about 5 mm apart, about 3 mm to about 4 mm apart, about 4 mm to about 9 mm apart, about 4 mm to about 8 mm apart, about 4 mm to about 7 mm apart, about 4 mm to about 6 mm apart, about 4 mm to about 5 mm apart, about 5 mm to about 9 mm apart, about 5 mm to about 8 mm apart, about 5 mm to about 7 mm apart, about 5 mm to about 6 mm apart, about 6 mm to about 9 mm apart, about 6 mm to about 8 mm apart, about 6 mm to about 7 mm apart, about 7 mm to about 9 mm apart, about 7 mm to about 8 mm apart, or about 8 mm to about 9 mm apart.

A biological pacemaker agent may be delivered to any of a variety of locations. For example, a biological pacemaker agent may be delivered to the myocardial muscle tissue and/or connective tissue of the heart. A biological pacemaker agent may be delivered to ventricular and/or atrial tissue. Biopacing interventions may be administered to various regions of the heart, including, but not limited to, the right ventricle, left ventricle, right atrium, left atrium, Bachman's bundle, and/or SA node.

The methods, systems, devices, and apparatus described herein are particularly useful for the delivery of any of a wide variety of biological pacemaker agents for the modulation of cardiac contraction and the treatment of cardiac disorders. A biological pacemaker agent may also be referred to herein, for example, as a “biological agent affecting cardiac pacing,” “biologic pacemaker agent,” “biopacemaker agent,” “biopacer agent,” “biopacing agent,” “biologic agent,” or “biological agent.”

Biological pacemaker agents include gene therapy or cell based therapies. A biological pacemaker agent may be exogenous, that is obtained form a source other than the tissue to which it is delivered.

A biological pacemaker agent includes genetic constructs, genetically engineered cells, or unmodified cells. A biological pacemaker agent provides pacemaking activity to cardiac cells, increasing or decreasing the intrinsic pacing rate of such cells. Cell based therapies include, but are not limited to, therapies based on the administration of stem cells, including, but not limited to induced pluripotent stem cells or genetically engineered cells. Gene therapy includes the delivery of a modified or unmodified polynucleotide. A polynucleotide may also be referred to herein as “polynucleotide sequence,” “nucleic acid,” “nucleic acid sequence,” “nucleotide sequence,” and similar terms. As used herein, the terms “encodes,” “encoding,” “coding sequence,” and similar terms refer to a nucleic acid sequence that is transcribed (in the case of DNA) and translated (in the case of mRNA) into a polypeptide in vitro or in vivo when placed under control of appropriate regulatory sequences. Polynucleotides can be made by traditional PCR-based amplification and known cloning techniques. Alternatively, a polynucleotide can be made by automated procedures that are well known in the art. A polynucleotide may include a start codon to initiate transcription and a stop codon to terminate translation. A polynucleotide may include one or more regulatory elements. A regulatory sequence is a nucleotide sequence that regulates expression of a coding sequence to which it is operably linked. Non-limiting examples of regulatory sequences include promoters, enhancers, transcription initiation sites, translation start sites, translation stop sites, and transcription terminators.

In some embodiments, a regulatory element includes a promoter region and a wide variety of promoters are known. Promoters act as regulatory signals that bind RNA polymerase in a cell to initiate transcription of a downstream (3′ direction) coding region. The promoter used may be a constitutive or an inducible promoter. It may be, but need not be, heterologous with respect to the host cell. In some aspects, tissue-specific promoters may be used. Tissue-specific expression may enhance the safety of a therapy described herein as expression in non-target tissue becomes less likely. For example, cardiac tissue specific promoters allow cardiac myocyte specific expression of the coding region of interest (including expression in stem cells with cardiac phenotype). Examples of cardiac tissue specific promoters include, but are not limited to, promoters from the following coding regions: an α-myosin heavy chain coding region, e.g., a ventricular α-myosin heavy chain coding region, β-myosin heavy chain coding region, e.g., a ventricular β-myosin heavy chain coding region, myosin light chain 2v coding region, e.g., a ventricular myosin light chain 2 coding region, myosin light chain 2a coding region, e.g., a ventricular myosin light chain 2 coding region, cardiomyocyte-restricted cardiac ankyrin repeat protein (CARP) coding region, cardiac α-actin coding region, cardiac m2 muscarinic acetylcholine coding region, ANP coding region, BNP coding region, cardiac troponin C coding region, cardiac troponin I coding region, cardiac troponin T coding region, cardiac sarcoplasmic reticulum Ca-ATPase coding region, and skeletal α-actin coding region. Further, chamber-specific promoters or enhancers may also be employed, e.g., for atrial-specific expression, the quail slow myosin chain type 3 (MyHC3) or ANP promoter may be used. Examples of ventricular myocyte-specific promoters include a ventricular myosin light chain 2 promoter and a ventricular myosin heavy chain promoter. Other useful promoters, for example, would be sensitive to electrical stimulus that could be provided from, for example, an implantable device. Electrical stimulation can promote gene expression (Padua et al., U.S. Patent Application No. 2003/0204206 A1).

Other regulatory regions include drug-sensitive elements (e.g., a drug-inducible suppressor or promoter). Drug-responsive promoters may induce or suppress expression of an operably linked coding region. For example, a tetracycline responsive element (TRE) that binds doxycycline may present within a promoter construct. When doxycycline is removed, transcription from the TRE is turned off in a dose-dependent manner. Examples of inducible drug-responsive promoters are the ecdysone-inducible promoter (Johns and Marban, U.S. Pat. No. 6,214,620) and rapamycin-dependent expression (Clackson et al., U.S. Pat. No. 6,506,379, see also Discher et al., 1998, J. Biol. Chem; 273:26087-26093; Prentice et al., 1997, Cardiovascular Res; 35: 567-576).

Further examples of regulatory regions include enhancers, such as cardiac enhancers, to increase the expression of an operably linked coding region in cardiac tissue, such as regions of the cardiac conduction system. Such enhancer elements may include the cardiac specific enhancer elements derived from Csx/Nkx2.5 regulatory regions (Lee and Izumo, U.S. Patent Application 2002/0022259) or the cGATA-6 enhancer.

An expression vector may optionally include a ribosome binding site and a start site (e.g., the codon ATG) to initiate translation of the transcribed message to produce the polypeptide. It may also include a termination sequence to end translation. A termination sequence is typically a codon for which there exists no corresponding aminoacetyl-tRNA, thus ending polypeptide synthesis. The polynucleotide used to transform the host cell may optionally further include a transcription termination sequence.

Suitable polynucleotides for use with the invention may be obtained from a variety of sources including, without limitation, GenBank (National Center for Biotechnology Information (NCBI)), EMBL data library, SWISS-PROT (University of Geneva, Switzerland), the PIR-International database; and the American Type Culture Collection (ATCC), 10801 University Boulevard, Manassas, Va. 20110-2209.

Any suitable vector or delivery vehicle may be utilized to transfer the desired nucleotide sequence to the targeted cardiac cells. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to those of skill in the art.

A vector may be an expression vector. In various embodiments, the expression vectors are packaged into viruses and are delivered in proximity to targeted cells, tissue or organs. Suitable viral vectors include, but are not limited to, retroviral vectors, lentiviral vectors, adenoviral vectors, adeno-associated viral vectors, vaccinia viral vectors, and Semliki Forest viral vectors. For example, an expression vector may be packaged into adenoviruses, such as helper-dependent adeno viral vector (HDAd) or adeno-associated virus pseudo-type 9 (AAV2/9). HDAd virus packaging typically elicits less of an immunogenic response in vivo compared to some other adenoviruses and thus allows for longer term expression. AAV2/9 packaging can result in cardiac tropism as well as a prolonged expression time frame. Other viruses of clinical relevance include lentiviruses. Replication deficient lentiviruses are RNA viruses, which can integrate into the genome and lead to long-taint functional expression. Viral vectors systems in addition to lentiviral vectors, AAV vectors, and HD AdV may also be used for the delivery of a polynucleotide encoding an ion channel. Alternatively, non-viral delivery systems may be employed. For example, liposomes, DNA complexes, plasmid, liposome complexes, naked DNA, DNA-coated particles, or polymer based systems may be used to deliver the desired sequence to the cells.

A biological pacemaker agent may include a polynucleotide encoding, for example, an ion channel, including, but not limited to, calcium channel, a sodium channel, a chloride channel, a hyperpolarized activated cyclic nucleotide (HCN) channel, SERCA2a, a non-specific leak channel, a voltage-gated ion channels, or a potassium channel. A potassium ion channel may be one of a large family of mammalian potassium channels, such as for example, Kv1 (shaker), Kv2, Kv3 (Shaw), Kv4 (Shal), Kv5, Kv6, Kv7, Kv8, or Kv9. In one embodiment, the potassium ion channel is Kv1.3. In some aspects, a polynucleotide may encode a modified ion channel. Such modifications include, but are not limited to, modifications that alter, add, or delete one or more amino acids or the ion channel polypeptide, modifications that encode a truncated ion channel polypeptide, and/or modifications that encode a fusion product of an ion channel polypeptide, or portion thereof, and another polypeptide sequence.

Hyperpolarization-activated cyclic nucleotide-gated channels (HCN) serve as ion channels across the plasma membrane of heart and brain cells and are sometimes referred to as “pacemaker channels” because they help to generate rhythmic activity within groups of heart and brain cells. HCN channels are encoded by four genes (HCN1-4). A polynucleotide may encode, for example, HCN1, HCN2, HCN3, or HCN4. See, for example, WO 2005/062958. In some aspects, a polynucleotide may encode a modified HCN ion channel. Such modifications include, but are not limited to, modifications that alter, add, or delete one or more amino acids or the ion channel polypeptide, modifications that encode a truncated ion channel polypeptide, and/or modifications that encode a fusion product of an ion channel polypeptide, or portion thereof, and another polypeptide sequence. See, for example, U.S. Published Application 20090099611, Jaye et al., 2010, Circulation; 122(21-supplement):A21428, and Zeng et al., 2010, Circulation; 122(21-supplement):A18147.

In some aspects, the HCN channel is an HCN4 channel, such as, for example, a human, mouse, or rat HCN4. In some aspects, an HCN4 polypeptide may include additional mutations in the region linking the S3 and S4 segments, including, but not limited to, any of those described in Sigg et al., U.S. Patent Application Publication 2009/0099611. In some aspects, an HCN4 polypeptide may include one or more of the truncation mutations described in U.S. Provisional Patent Application Ser. No. 61/351,836; “Compositions and Methods to Treat Cardiac Pacing Conditions;” filed Jun. 4, 2010.

A biological pacemaker agent may be delivered to a delivery site by any of a variety of methods, including, for example, injection, infusion, instillation, topical application, delivery by a needle, and/or delivery by a catheter. Once delivered to a delivery site, a genetic construct may be introduced to the myocardium or other tissue using any suitable technique. A polynucleotide encoding a biological pacemaker agent or a vector including a polynucleotide encoding a biological pacemaker agent can be delivered into a cell by, for example, transfection or transduction procedures. Transfection and transduction refer to the acquisition by a cell of new genetic material by incorporation of added nucleic acid molecules. Transfection can occur by physical or chemical methods. Many transfection techniques are known to those of ordinary skill in the art including, without limitation, calcium phosphate DNA co-precipitation, DEAE-dextrin DNA transfection, electroporation, naked plasmid adsorption, and cationic liposome-mediated transfection (commonly known as lipofection). Transduction refers to the process of transferring nucleic acid into a cell using a DNA or RNA virus. Suitable viral vectors for use as transducing agents include, but are not limited to, retroviral vectors, adeno-associated viral vectors, lentiviral vectors, herpes simplex viral vectors, vaccinia viruses, and Semliki Forest virus vectors.

Delivery may be accomplished with the use of delivery devices and systems. For example, delivery may be accomplished with a needle or catheter. In some embodiments, such devices and systems may be used for epicardial delivery. In some embodiments, such devices and systems may be used for endocardial delivery. In some embodiments, a device or system may have electric sensing capabilities, for example, electrodes for sensing electric activity and delivering pacing stimuli in order to determine the desired location for delivery of a biological pacemaker agent. Once the location is determined, genetically engineered viruses, gene-modified cells or unmodified cells are delivered to the myocardium at that location to form a biological pacemaker. A delivery device or system may include an injection device that injects the vectors, viruses or cells into the myocardium.

For example, a catheter system may be used for the delivery of a biological pacemaker agent affecting cardiac pacing to two or more sites in the heart, the catheter system including a catheter suitable for endocardial access and a needle suitable for delivery to two or more sites in the heart, the needle extendable through a distal tip of the catheter. In some aspects of the catheter system, the needle includes two or more openings, periodically spaced. In some aspects of the catheter system, the needle includes a single opening at the distal end of the needle. In some aspects, the catheter system further includes an electrode for recording electrical impedance indicating that the needle remains located within myocardial tissue. In some aspects, the catheter system further includes image guidance technology to record the site of delivery of each biological agent affecting cardiac pacing in cardiac tissue.

For example, a system for the delivery of a biological pacemaker agent affecting cardiac pacing to two or more sites in the heart may include a catheter suitable for endocardial access that includes a catheter body that defines an inner lumen and a needle for placement within the inner catheter lumen, the needle delivering a fluid including a biological pacemaker agent affecting cardiac pacing to two or more sites in the heart, delivery sites periodically spaced, for example, about less than about 10 millimeters (mm) from any other delivery site, and the delivery sites forming a linear pattern that is perpendicular to the fibers of the heart. In some aspects, the system further includes a fluid including a biological pacemaker agent affecting cardiac pacing. In some aspects, the system further includes at least one electrode for recording electrical impedance indicating that the needle remains located within myocardial tissue. In some aspects, the system further includes image guidance technology to record the site of delivery of each biological pacemaker agent affecting cardiac pacing in cardiac tissue. In some aspects, the needle includes two or more openings, the openings having a periodic spacing of 10 mm or less along the distal end of the needle. In some aspects, the needle includes single opening at the distal end of the needle.

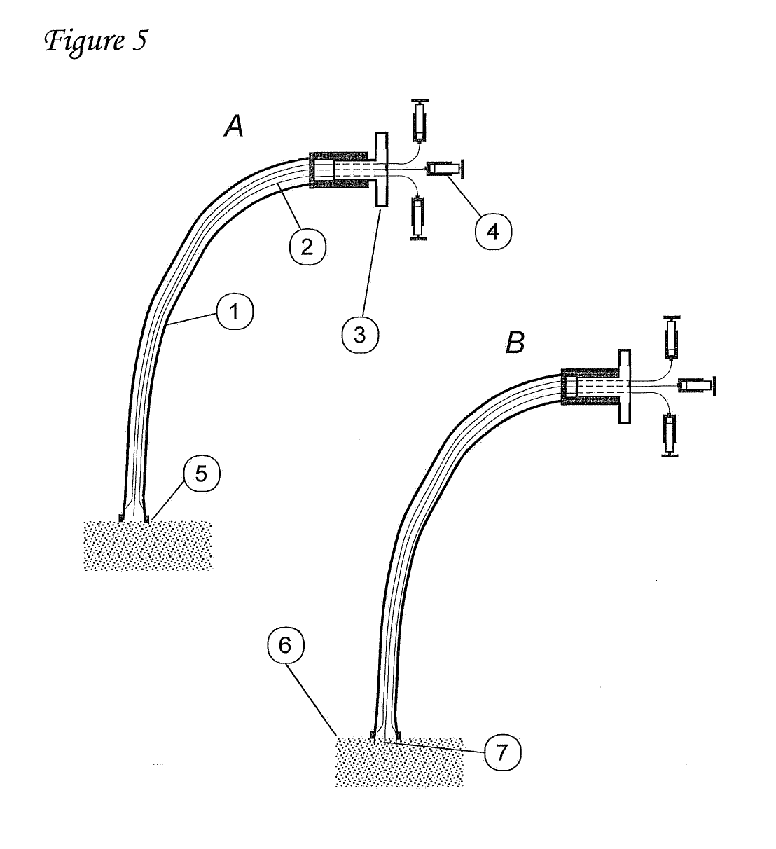

An example of a delivery device is shown in FIG. 5. This figure shows a catheter with multiple needles that are spaced the correct distance for delivery of a biological pacemaker agent in a linear pattern to tissue (6). Such a catheter may include a catheter body (1), needle body (2), needle deploying handle, one or more syringes to hold biologic pacemaker or other intervention for delivery to tissue (4), and one or more needles (7). In FIG. 5A, needles are retracted prior to delivery by injection. In FIG. 5B, needles are shown deployed into the tissue. In some embodiments, the device includes one or more ring electrodes (5) that may be used once a needle is placed in tissue to record EGM signal, the ring serving as the return electrode. The use of such a ring electrode helps determine that needles have made contact with the tissue before a biological pacemaker agent is delivered.

Needles may be preformed. Needles may be of materials such as appropriate for medical applications, for example, nickel titanium (nitinol). In FIG. 5, three needles are shown, but a device as described herein may include a single needle or multiple needles for delivery of a biological pacemaker agent to two or more sites in a tissue so that the delivery sites form a linear pattern in the tissue. When multiple needles are present in a device or system, two, three, four, five, six, seven, eight, nine, ten, or more needles may be present. With multiple needles, needles may be arranged in a pattern constellation that allows for the delivery of a biological pacemaker agent and/or other intervention agent, in a linear pattern such that deliver sites are spaced so that as an agent disperses from a given delivery site it overlaps with the agent dispersing from neighboring delivery sites, forming a single mass. For example, delivery may be spaced about 1 mm to about 10 mm apart. In some embodiments, a single needle may be used repeatedly for delivery to two, three, four, five, six, seven, eight, nine, ten, or more sites in a linear pattern. Such delivery may be in conjunction with the use of a navigation system.

A needle may have two or more openings, the openings having a periodic spacing of 10 mm or less along the distal end of the needle, and the biological pacemaker agent affecting cardiac pacing is delivered through all needle openings simultaneously.

A needle may have a single opening at the distal end of the needle, the biological pacemaker agent affecting cardiac pacing is repeatedly delivered through the distal end opening of the needle, the needle is withdrawn or advanced between each delivery, and delivery of each biological pacemaker agent affecting cardiac pacing is in a linear pattern and each delivery is spaced 10 mm or less apart.

Delivery may be accomplished with the use of an automated/robotic system. Such an automatic system may include any one of more of the aspects of a biological pacemaker agent, delivery method, and/or delivery tool or device as described herein. Such an automated system may be used, for example, for epicardial delivery, endocardial delivery, and/or delivery via a tangential injection approach in which the needle penetrates along the tissue wall. Such an automated system may draw one or more needles at a programmable rate. Such an automated system may inject a biological pacemaker agent and/or other intervention agent at a programmable rate and/or volume.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein are applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein.

The present invention and/or one or more portions thereof include circuitry that may include a computer system operable to execute software to provide for the determination of a physiological state, e.g., bradycardia and heart failure. Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and most any device capable of providing suitable computing capabilities may be used according to the present invention. Further, various peripheral devices, such as a computer display, a mouse, a keyboard, memory, a printer, etc., are contemplated to be used in combination with a processing apparatus in the computer system.

In view of the above, it will be readily apparent that the functionality as described herein may be implemented in any manner as would be known to one skilled in the art.

In the context of the heart, any conventional or developed methods for detecting modulation of the cells of the heart by electrophysiological assay may be used to monitor the establishment of an artificial pacemaker. For example, the modulation of cardiac electrical properties may be observed by determining cardiac action potential characteristics, such as action potential duration (APD), or by performing a conventional electrocardiogram (ECG) before and after administration of the expression vector and inspecting the ECG results. ECG patterns from a heart's electrical excitation have been well studied. Various methods are known for analyzing ECG records to measure changes in the electrical potential in the heart associated with the spread of depolarization and repolarization through the heart muscle.

As used herein, unless the context makes clear otherwise, “treatment,” and similar word such as “treated,” “treating” etc., is an approach for obtaining beneficial or desired results, including and preferably clinical results. Treatment can involve optionally either the amelioration of symptoms of the disease or condition, or the delaying of the progression of the disease or condition. As used herein, an “effective amount” or a “therapeutically effective amount” of a substance is that amount sufficient to affect a desired biological effect, such as beneficial results, including clinical results. In some embodiments, more than one biological pacemaker agent may be administered. In some embodiments, one or more biological pacemaker agent may be administered in conjunction with additional therapeutic agents.

Therapeutically effective concentrations and amounts may be determined for each application herein empirically by testing the compounds in known in vitro and in vivo systems, such as those described herein. Dosages for humans or other animals may then be extrapolated therefrom. With the methods of the present invention, the efficacy of the administration of one or more interventions may be assessed by any of a variety of parameters well known in the art.

As used herein, the term “subject” includes, but is not limited to, humans and non-human vertebrates. In preferred embodiments, a subject is a mammal, particularly a human. A subject may be an individual. A subject may be a patient. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

The methods described herein may include in vitro, ex vivo, or in vivo methods. As used herein “in vitro” is in cell culture and “in vivo” is within the body of a subject. With the present invention, an isolated biological pacemaker may be delivered. As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

The methods and apparatus described herein may be used with any of a wide variety of additional interventions that influence cardiac pacing, such as, for example, ablation, and/or drug delivery. In some aspects, the drug is a pharmaceutical drug. In some aspects, the drug is a biological agent, such as for example, a polypeptide, including, but not limited to, an antibody.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Dispersal of Gene Expression Following Cardiac Injection

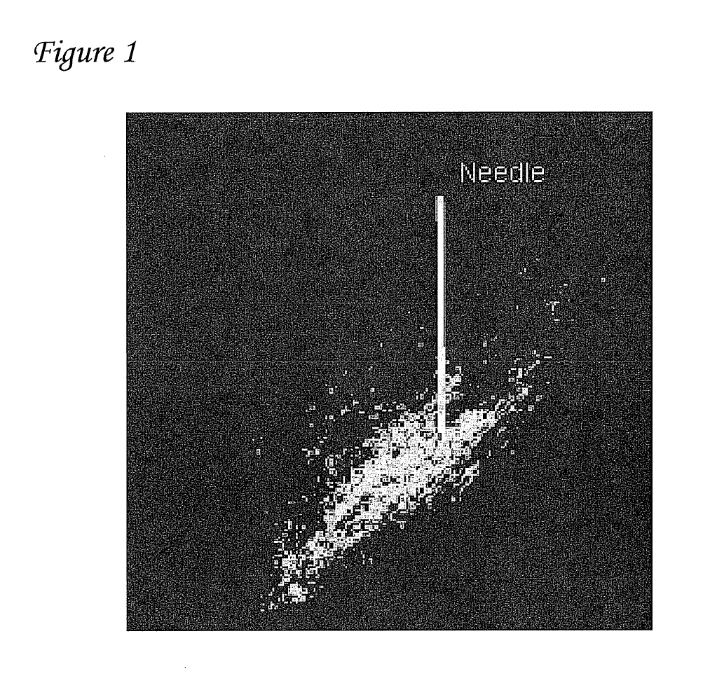

A single injection of ˜100 μ 1 biologic volume of gene constructs expressing green fluorescent protein (GFP) or luciferase were injected into the left ventricle of pigs. Gene expression and dispersion of gene expression were monitored by fluorescent microscopy of histological samples. A representative sample is shown in FIG. 1. The white bar in FIG. 1 depicts the needle and is 10 millimeters (mm) long. As shown in FIG. 1, it was found that the gene disperses over a distance of ˜10 mm.

From these results it was determined that injections performed with a distance of about 2-5 mm between injections should be used to obtain optimal biopacemaker function. The asymmetric distribution of expression is likely because biologic solutions tend to disperse along the fiber direction. This information is critical and is taken into account during the biological injections. The transfected region that results from linear injections performed perpendicular to the fiber direction results in a single overlapping region of gene expression and forms a linear pacemaker structure in the heart.

### Example 2

### Delivery of Biological Pacemaker Addressing Source-Sink Mismatch

Several efforts have been undertaken to create an artificial site in the heart that can mimic the pacemaking function of the SA nod. However, none of these studies have been able to demonstrate a stable biological pacemaker. See, for example, Qu et al., 2003, Circulation; 107(8):1106-9; Bucchi et al., 2006, Circulation; 114(10):992-9; Tse et al., 2006, Circulation; 114(10):1000-11; and Kashiwakura et al., Circulation; 114(16):1682-6. This variation in data and biopacemaking activity may be because these investigators overlooked something critical in the design of the biological pacemaker—the source-sink mismatch. With source-sink mismatch, if the tissue load on the regions driving the excitation (i.e. pacemaker region) is large relative to pacemaker's size, the pacemaker is unable to drive the tissue in a stable and reproducible manner. And, to achieve reliable excitation of the load tissue and produce stable pacemaking, the pacemaker regions must be sufficiently large and of correct geometry relative to the tissue architecture (e.g. fiber direction).

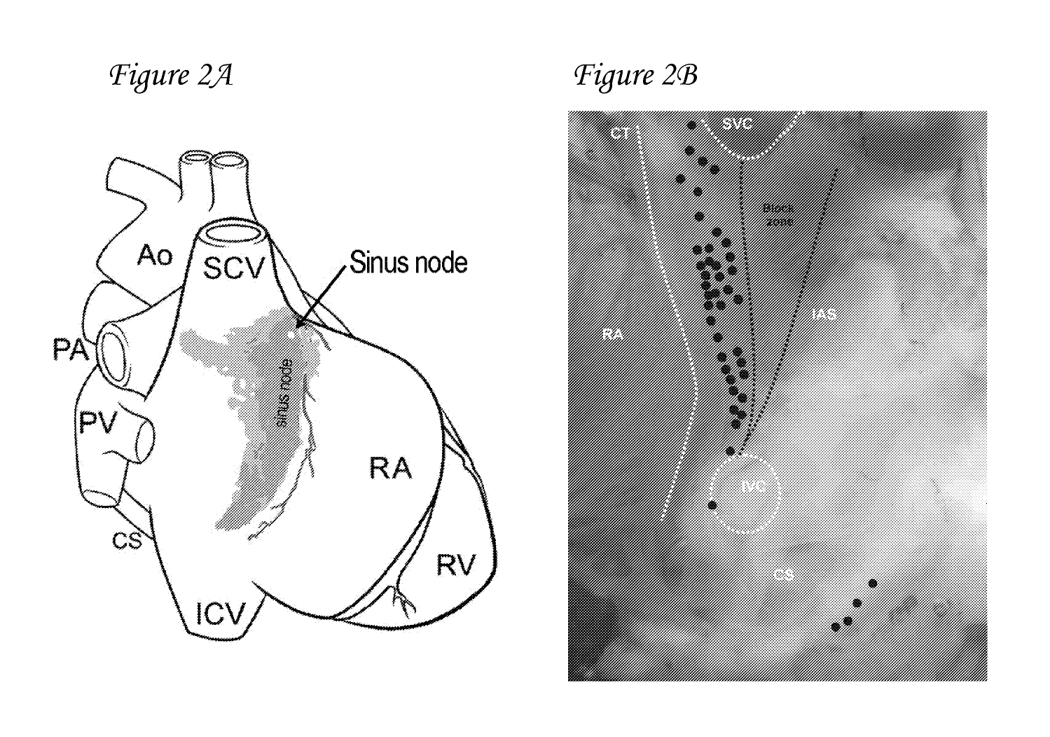

The sinus node (SN), the primary cardiac pacemaker, is complex and heterogeneous. As shown in FIGS. 2A and 2B, the sinus node is a large linear structure. The extent of the sinus node can be almost 2 centimeters or longer. Functionally the site of earliest activation hops from place to place over the extent of the SA node (e.g. with changes in autonomic tone). A long and linear design is nature's answer to address the source-sink mismatch problem. Simply stated, this problem is inability of a small source to excite and drive a large load such as the atrial muscle. To be able to drive a large load the source must also be of reasonable size. A linear source would be the best way to solve this problem without making the structure unusually large. Alternatively, a much larger mass of cells would be needed if the same problem is solved using a circular mass of pacemaker cells.

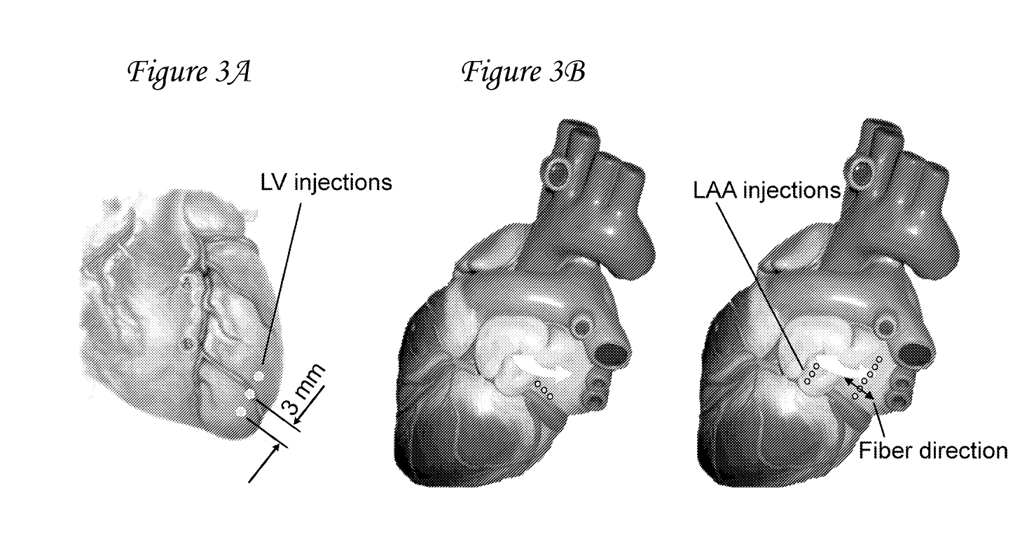

This example demonstrates that a stable and robust pacemaker is generated by the generation of a linear artificial pacemaker. To accomplish this, three closely spaced injections of a biopacemaker gene construct were performed. Injection was in a linear pattern and the linear pattern is perpendicular to the fiber direction of the heart. The spacing of the injections was adjusted such that as each of the injections disperses, the distribution of the injections overlaps to generate one single large mass of transfected cells. This ensures that the resulting structure is large and robust functionally. This linear injection strategy is shown in FIGS. 3A and 3B. FIG. 3A presents the pattern of injection in the left ventricle (LV) and FIG. 3B presents the pattern of injection in the left atrium (LA).

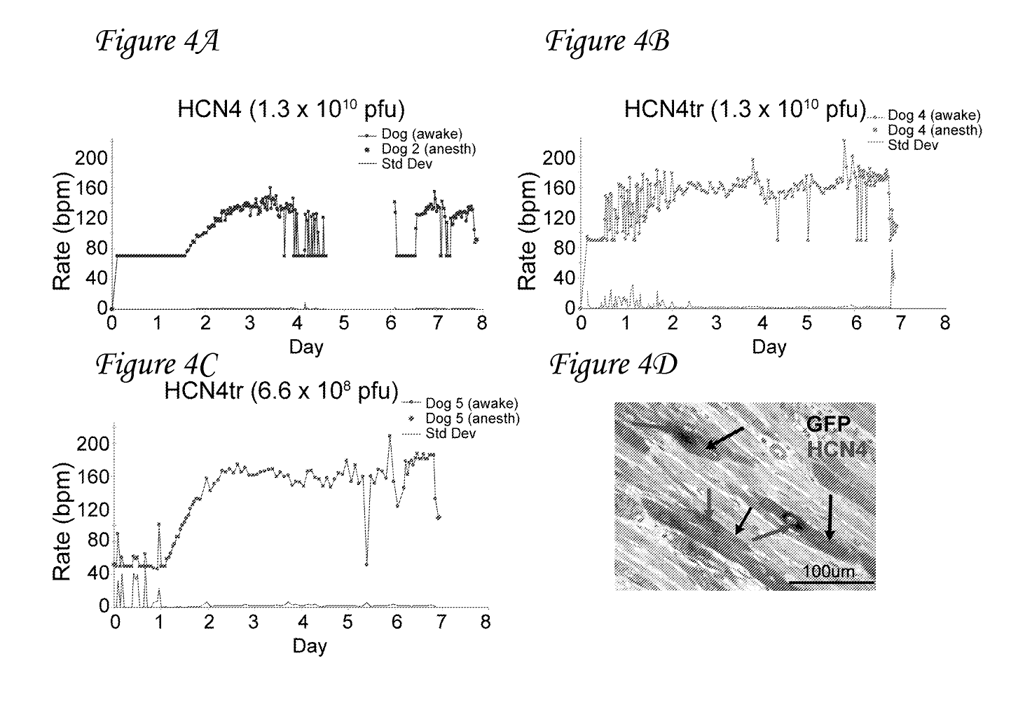

Using this strategy, experiments were performed in which a HCN4 pacemaker gene constructs were injected using a linear injection strategy into the left ventricles of three AV node ablated canines. In dog 2 (FIG. 4A) a dose of 1×10¹⁰ pfu of an adenovirus viral vector encoding for HCN4 wild type was injected. In dog 4 (FIG. 4B) a dose of 1.3×10¹⁰ pfu of an adenovirus viral vector encoding for a HCN4 truncated gene construct (“HCN4tr” as described in U.S. Published Application 20090099611) was injected. And, in dog 5 (FIG. 4C) a dose of 6.6×10⁸ pfu of the adenovirus viral vector encoding the HCN4 truncated HCN4tr construct was injected. From the gene dispersion experiments in Example 1, it was determined that the gene disperses over a distance of ˜10 mm (see FIG. 1). Thus, three injections were performed with a spacing between injections of about 2 mm to about 5 mm.

The results from three AV node ablated canines in which the left ventricle was injected with HCN4 pacemaker gene at three sites using a linear injection strategy are presented in FIGS. 4A-4C. In each animal a significant increase in ventricular heart rate was observed in a time dependent manner; heart rate increased as the protein expression of HCN4 ion channel increased with time. FIG. 4D demonstrates expression of the HCN protein using immunohistochemical techniques. The pacemaking activity was very stable and showed very little variation over the 15 second (s) window that was used to periodically collect snippets of ventricular rhythm using an implanted device.

This example demonstrates that both the spacing and geometry of injections are important in creating a stable biological pacemaker. The geometry of a linear injection addresses the source-sink mismatch problem and creates a stable biological pacemaker.

### Example 3

### Endocardial Delivery

Example 2 used an epicardial approach in which the access to the animal's heart was obtained by performing a thoracotomy. However, the same strategy can be implemented using an endocardial approach. With such an endocardial approach, an image guidance system may be used. When an image guidance system is used, each injection may be marked on the screen. Subsequent injections are to be placed about 2 mm to about 5 mm adjacent to preceding injections, in a linear pattern perpendicular to the fibers of heart tissue.

A needle catheter system that performs a linear injection endocardially may be used for the delivery of a biopacemaker gene. Such a needle catheter system may be a multi-needle catheter system. A tangentially approach in which needle is inserted tangential to the myocardium may also be used.

In such implementations, multiple electrodes along the length of the needle may be used to ensure that it is within the myocardium during injections. The measured impedance between each electrode and a distant return electrode is distinct when the needle is in the tissue or outside in the cavity. This impedance information can be used to assess if the needle is suitably inside the myocardium.

The needle can have an end hole and can be withdrawn as multiple small volume injections are performed along the needle tract. Alternatively, side holes along the desired length (3-4 cm) at periodic spacing (about 2 mm, about 3 mm, about 4 mm, about 5 mm, to about 6 mm) can be placed so that the biologic can be injected all along the needle length simultaneously. In this case tiny impedance measuring electrodes will be placed adjacent to each of the holes so that it can be determined that the tissue contact along the entire length of the needle is uniform. In absence of this feedback, the biologic delivery may be non-uniform along the needle's length.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. All headings are for the convenience of the reader and should not be used to limit the meaning of the text that follows the heading, unless so specified.

What is claimed is:

1. A method for delivering a biological agent affecting cardiac pacing to the heart, the method comprising:
delivering the biological agent affecting cardiac pacing at two or more sites in the heart;
and the delivery sites forming a linear pattern that is perpendicular to the fibers of the heart.

2. The method of claim 1, wherein the delivery of the biological agent affecting cardiac pacing is into cardiac atrial cells or cardiac ventricle cells.

3. The method of claim 1, wherein the delivery sites are perpendicular to the fibers of the heart.

4. The method of claim 1, wherein each delivery site located less than about 10 millimeters (mm) from any other delivery site.

5. The method of 1, wherein the delivery of the biological agent affecting cardiac pacing comprises two, three, four, five, six, or more delivery sites within the heart.

6. The method of claim 1, the biological agent affecting cardiac pacing comprising cell therapy.

7. The method of claim 6, cell therapy comprising stem cell therapy or genetically modified cell therapy.

8. The method of 1, the biological agent affecting cardiac pacing comprising an exogenous polynucleotide encoding a membrane polypeptide that regulates the flow of ions across a cell membrane.

9. The method of claim 8, wherein the polynucleotide is present in a vector.

10. The method of claim 8, wherein the exogenous polynucleotide encoding a membrane polypeptide that regulates the flow of ions across a cell membrane is present in a genetically modified cell.

11. The method of claim 8, wherein the membrane polypeptide that regulates the flow of ions across a cell membrane is an ion channel.

12. The method of claim 11, wherein the ion channel comprises a potassium channel.

13. The method of claim 12, wherein the potassium channel comprises a member of the Kv1, Kv2, Kv3, Kv4, Kv5, Kv6, Kv7, Kv8, or Kv9 family.

14. The method of claim 11, wherein the ion channel comprises a hyperpolarization-activated cyclic nucleotide-gated (HCN) channel.

15. The method of claim 14, wherein the hyperpolarizaton-activated cyclic nucleotide-gated (HCN) channel comprises HCN1, HCN2, HCN3, or HCN4.

16. The method of claim 14, wherein the amino acid sequence of the encoded HCN polypeptide comprises one, two, three, four, five, six, or more mutations.

17. The method of claim 14, wherein the amino acid sequence of the HCN polypeptide comprises a truncation.

18. The method of claim 1, wherein the delivering comprises use of a needle.

19. The method of claim 1, wherein the delivering comprises injection.

20. The method of claim 1, wherein the delivering comprises use of a catheter.

21. The method of claim 1, wherein delivering comprises epicardial delivery.

22. The method of claim 1, wherein the delivering comprises endocardial delivery.

23. The method of claim 1, further comprising the use of image guidance technology to record the site of delivery of each biological agent affecting cardiac pacing in cardiac tissue.

24. The method of claim 1, further comprising recording electrical impedance to determine that the needle remains located within myocardial tissue.

25. A catheter system for the delivery of a biological agent affecting cardiac pacing to two or more sites in the heart, the catheter system comprising:
a catheter suitable for endocardial access;
one or more needles suitable for delivery to two or more sites in the heart, the delivery sites forming a linear patter; and
the needle extendable through a distal tip of the catheter.

26. The catheter system of claim 25, further comprising an electrode for recording electrical impedance indicating that the needle remains located within myocardial tissue.

27. The catheter system of claim 25, further comprising image guidance technology to record the delivery sites of each biological agent affecting cardiac pacing in cardiac tissue.

28. A system for the delivery of a biological agent affecting cardiac pacing to two or more sites in the heart, the system comprising:
a catheter suitable for endocardial access that includes a catheter body that defines an inner lumen;
a needle for placement within the inner catheter lumen; and
the needle delivers a fluid comprising a biological agent affecting cardiac pacing to two or more sites in the heart, the delivery sites forming a linear pattern.

29. The system of claim 28, further comprising a fluid comprising a biological agent affecting cardiac pacing.

30. The system of claim 28, further comprising at least one electrode for recording electrical impedance indicating that the needle remains located within myocardial tissue.

31. The system of claim of claim 28, further comprising image guidance technology to record the delivery sites of the biological agent affecting cardiac pacing in cardiac tissue.
